(19) United States
(12) Patent Application Publication
(10) Pub. No.: US 2004/0126392 A1
(43) Pub. Date: Jul. 1, 2004

(54) Cancer preventive agent and food

(76) Inventor: Kenichi Matsunaga, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/695,762

(22) Filed: Oct. 30, 2003

(30) Foreign Application Priority Data
Dec. 27, 2002 (JP) 2002-381275

(51) Int. Cl. A61K 35/84

(52) U.S. Cl. 424/195.15

(57) ABSTRACT

A cancer preventive agent and food are disclosed, which contain Tricholoma matsutake, in particular Tricholoma matsutake of the FERM BP-7304 strain, and any of mycelia, broths, or fruit bodies (including spores) thereof, as they are, dried products thereof, or extracts thereof (e.g., a hot water extract or an alkaline solution extract). Methods of preventing a cancer by the use of the cancer preventive agent and food are also disclosed.

### BACKGROUND OF THE INVENTION

1. Field of the Invention

The present invention relates to a cancer preventive agent and food, which prevent the occurrence of cancer in animals and humans. The cancer preventive agent and food of the present invention may be administered not only as a medicament but also in various forms, for example, as eatable and drinkable products such as health-promoting foods (specified health food and nutritional-functional food), as so-called health food (both including drinkable products), or as feeds. Further, the agent of the present invention may be administered in the form of an agent that is temporarily kept in the mouth but then spat out without the retention of most components, for example, a dentifrice, a mouthwash agent, a chewing gum, or a collutorium, or in the form of an inhalant drawn in through the nose.

2. Description of Related Art

Mushrooms have contributed to the health of Japanese people throughout history, due to their various physiological activities. At present, formulations of polysaccharides derived from mushrooms are used as antitumor medicaments, and clinical effects have been revealed based on scientific evidence.

For example, with respect to matsutake [Tricholoma matsutake (S. Ito & Imai) Sing.], JP-B-57-1230(Kokoku) discloses that emitanine-5-A, emitanine-5-B, emitanine-5-C, and emitanine-5-D, which are separated and purified from a liquid extract obtained by extracting a liquid culture of Tricholoma matsutake mycelia with hot water or a diluted alkaline solution, exhibit activity of inhibiting the proliferation of sarcoma 180 cells. Further, JP Patent No. 2767521 discloses that a protein with a molecular weight of 0.2 to 0.21 million (a molecular weight of a subunit=0.1 to 0.11 million) that is separated and purified from an extract of Tricholoma matsutake fruit bodies with water exhibits antitumor activity. In this way, it has been reported that the extract of T. matsutake is recognized to have therapeutic effects against cancer (cancer cell proliferation inhibition and antitumor activity).

Meanwhile, cancer prevention completely differs from its therapy. It has been reported that krestin (PSK) or an extract or the like of Polyprus frondosus among mushrooms has cancer prevention activities, and it is considered that immunoregulation or antioxidant action is involved in a complex manner in the obtainment of the effects. The mechanism of the occurrence of cancer still has many points that remain unrevealed, and therefore various types of research and development are actively carried out regarding not only therapeutic means for treating cancers that have occurred but also means for preventing the occurrence of cancer.

### SUMMARY OF THE INVENTION

An object of the present invention is to elucidate the mechanism of cancer occurrence and, based on the elucidated fact, provide a cancer preventive agent using mushrooms.

Another object of the present invention is to elucidate the mechanism of cancer occurrence and, based on the elucidated fact, provide a cancer preventive food using mushrooms.

Still another object of the invention is to provide a method of preventing a cancer by the administration of the cancer preventive agent.

A further object of the invention is to provide a method of preventing a cancer by the intake of the cancer preventive food.

The present invention relates to a cancer preventive agent containing Tricholoma matsutake or an extract thereof.

Further, the present invention relates to a cancer preventive food containing Tricholoma matsutake or an extract thereof.

Further, the present invention relates to a method of preventing a cancer which comprises administrating to a human or an animal in an effective amount of the cancer preventive agent.

Still further, the present invention relates to a method of preventing a cancer which comprises the intake of by a human or an animal in an effective amount of the cancer preventive food.

### BRIEF DESCRIPTION OF THE DRAWINGS

FIG. 1 is a graph showing chronological changes of body weights (of rats) through azoxymethane treatment in Examples.

FIG. 2 is a graph showing chronological changes of the amount of feed intake (of rats) through azoxymethane treatment in Examples.

FIG. 3 is a graph showing the degree of precancerous changes due to the intake of feed to which dried powder (CM6271) of mycelia of the T. matsutake FERM BP-7304 strain has been added in Examples.

FIG. 4 is a graph showing the degree of precancerous changes by intake of feed to which dried powder (CM6271) of mycelia of the T. matsutake FERM BP-7304 strain has been added in Examples.

### DETAILED DESCRIPTION OF THE INVENTION

Tricholoma matsutake [(S. Ito & Imai) Sing.] to be used for a cancer preventive agent and food of the present invention can be used in any form of mycelia, broths, or fruit bodies and they can be used in either a fresh or dried state. In the present invention, fruit bodies include spores. Further, extracts from these mycelia, broths, and fruit bodies, may be used for the present invention.

In the present invention, the T. matsutake FERM BP-7304 strain is particularly preferably used.

The T. matsutake FERM BP-7304 strain was previously filed by the present applicant as a novel strain (PCT WO 02/30440 A1), and was deposited on Sep. 14, 2000, at Independent Administrative Institution, National Institute of Advanced Industrial Science and Technology (former National Institute of Bioscience and Human-Technology, Agency of Industrial Science and Technology, Japan). This T. matsutake FERM BP-7304 strain was a mycelium passage strain obtained by cutting out a fruit body tissue from the T. matsutake CM 6271 strain harvested in Kameoka, Kyoto, Japan, and culturing the tissue in a test tube. The FERM BP-7304 strain has been maintained in Biomedical Research Laboratories, Kureha Chemical Industries Co., Ltd.

The fruit body of the T. matsutake FERM BP-7304 strain had a fruit body form identical to a T. matsutake fruit body described on plate pages 9 and 26 of “Genshoku-nihon shin-kinrui zukan (1)” (edited by Rokuya Imaseki and Tsuguo Hongo, published by Hoikusha in 1957).

The T. matsutake FERM BP-7304 strain can be subcultured in a slant Ebios agar medium. After mycelia of the T. matsutake FERM BP-7304 strain is inoculated in a plate Ebios agar medium, white mycelia densely grow in a radial pattern, forming a large colony. When the colony is observed with a scanning electron microscope, an uncountable number of branched mycelia with a thickness of 1 to 2 μm are present and sometimes projections with a size of several μm are present on the side of the mycelia. For mass cultivation of the mycelia of the strain, the mycelia are inoculated on a liquid medium and cultured by stationary cultivation, shaking cultivation, tank cultivation, or the like.

It should be noted that the T. matsutake FERM BP-7304 strain can be maintained by subculture or cultured mostly in the form of mycelia, but it may also exist in the form of fruit body.

The mycological characteristics of the T. matsutake FERM BP-7304 strain are described below.

(1) Cultural and Morphological Characteristics in Malt Extract Agar Medium:

White hyphae grew densely and radially, forming a colony. The diameter of the colony on the 30th day after inoculation was about 4 cm.

(2) Cultural and Morphological Characteristics in Czapeck Agar Medium, Oatmeal Agar Medium, Synthetic Mucor Agar Medium, and Phenoloxidase Reaction Assay Medium:

Almost no growth of hyphae was observed in any of the above media even after 1 month had passed since inoculation.

(3) Cultural and Morphological Characteristics in YpSs Agar Medium:

The T. matsutake FERM BP-7304 strain grew in a mat shape having a white gloss. On the 30th day after inoculation, the growth distance was about 5 mm.

(4) Cultural and Morphological Characteristics in Glucose Dry Yeast Agar Medium:

The T. matsutake FERM BP-7304 strain grew in a mat shape having a white gloss. On the 30th day after inoculation, the growth distance was about 2 mm.

(5) Optimum Growth Temperature and Growth Range:

In a 100-mL Erlenmeyer flask containing 10 mL of sterilized liquid medium (3% glucose, 0.3% yeast extract, pH 7.0), about 2 mg of seed fungi of the T. matsutake FERM BP-7304 strain was each inoculated and cultured at various temperatures of 5 to 35° C. On 28th day of incubation, fungus bodies were taken out from the flask, washed well with distilled water, and then dried for mass measurement. The results show that the mass of the fungus bodies linearly increased within the temperature range of 5 to 15° C. and leniently increased within the temperature range of 15 to 25° C. Almost no fungi grew at temperatures of 27.5° C. or more. The optimum temperature for growth is from 15 to 25° C.

(6) Optimum Growth pH and Growth Range:

Liquid media (3% glucose, 0.3% yeast extract) were adjusted with 1 mol/L hydrochloric acid or 1 mol/L potassium hydroxide so that the media having various pH levels from 3.0 to 8.0 were prepared to determine the pH for fungus body growth. Namely, each medium was sterilized with a filter, and 10 mL of the sterilized medium was dispensed into a 100-mL sterilized Erlenmeyer flask. About 2 mg of seed fungi of the T. matsutake FERM BP-7304 strain was inoculated in the flask and cultured at 22° C. Thereafter, fungus bodies were taken out from the flask, washed well with distilled water, and then dried for mass measurement. The results show that the pH growth limit for the fungus bodies was from 3.0 to 7.0 and the optimum pH for growth was 4.0 to 6.0.

(7) Formation of Zone Line by Dual Culture:

On an Ebios plate agar medium, a block (about 3 mm×3 mm×3 mm) of the T. matsutake FERM BP-7304 strain and each block (about 3 mm×3 mm×3 mm) of 13 kinds of known T. matsutake strains (for example, IFO 6915 strain; Institute for Fermentation Osaka) were placed with about 2 cm of distance between each strain, and cultured at 22° C. for 3 weeks. Thereafter, it was determined whether a zone line was formed on the boundary between two colonies among them.

The results show that the T. matsutake FERM BP-7304 strain did not form definite zone lines against all of the known T. matsutake strains (13 kinds). It is considered that no zone line is formed by dual culture between different strains of T. matsutake, and among the known T. matsutake strains (13 kinds) there was no combination of strains that formed a definite zone line therebetween. Therefore, it is considered the strains are compatible one another.

(8) Nutritional Requirement:

About 2 mg of seed fungi of the T. matsutake FERM BP-7304 strain was inoculated in a 100-mL Erlenmeyer flask containing 10 mL of sterilized synthetic medium for mycorrhizal fungus (Ohta medium, Ohta et al. “Trans. Mycol. Soc. Jpn.,” 31, 323-334, 1990), and cultured at 22° C. On 42nd day of culturing, fungus bodies were taken out from the flask, washed well with distilled water, and dried for mass measurement. Consequently, 441 mg of fungus body was obtained.

Instead of glucose in the above synthetic medium for mycorrhizal fungus as a carbon (C) source, any one of 28 kinds of carbohydrate-related substances was added to each medium. The T. matsutake FERM BP-7304 strain was inoculated and cultured on each medium, and after the completion of culture the mass of fungus bodies was measured. As a result, the carbohydrate-related substances are listed below in descending order corresponding to the fungus body mass:

Wheat starch>corn starch>dextrin>methyl β glucoside>cellobiose>mannose>fructose>arabinose>sorbitol>glucose>lactose>glycogen>mannitol>ribose>maltose>trehalose>galactose>raffinose>melibiose>N-acetylglucosamine.

Incidentally, almost no growth of the fungi was observed in cellulose, dulcitol, sucrose, xylose, methyl a glucoside, inulin, inositol, or sorbose.

Next, instead of ammonium tartrate in the above synthetic medium for mycorrhizal fungus as a nitrogen (N) source, any one of 17 kinds of nitrogen-related substances was added to each medium. The T. matsutake FERM BP-7304 strain was inoculated and cultured on each medium, and after the completion of culture the mass of fungus bodies was measured. As a result, the nitrogen-related substances are listed below in descending order corresponding to the fungus body mass:

Corn steep liquor>soy peptone>milk peptone>ammonium nitrate>ammonium sulfate>ammonium tartrate>ammonium carbonate>asparagine>ammonium phosphate>ammonium chloride>sodium nitrate>meat extract>yeast extract>casamino acid>chlorella>triptone>potassium nitrate.

Further, among minerals and vitamins in the above synthetic medium, a medium was prepared without a particular single component. The T. matsutake FERM BP-7304 strain was inoculated and cultured on that medium, and after the completion of culture the mass of fungus bodies was measured.

As a result, even when any one of calcium chloride•dihydrate, manganese (II) sulfate•pentahydrate, zinc sulfate•heptahydrate, cobalt sulfate•heptahydrate, copper sulfate•pentahydrate, nickel sulfate•hexahydrate, amine hydrochloride, nicotinic acid, folic acid, biotin, pyridoxine hydrochloride, carnitine chloride, adenine sulfate•dihydrate, and choline hydrochloride was removed from the medium, the fungus body mass was almost uneffected.

On the other hand, when any one of magnesium sulfate•heptahydrate, iron (II) chloride, and potassium dihydrogen phosphate was removed, the fungus body mass remarkably reduced. In other words, magnesium, iron, phosphorus, and potassium are considered essential for the growth of the T. matsutake FERM BP-7304 strain.

(9) DNA Base Composition (GC Content):

The GC content was 49.9%.

(10) DNA Pattern Prepared by RAPD Method:

In terms of DNA patterns prepared by the RAPD (Random Amplified Polymorphic DNA) method independently using 6 different kinds of PCR (Polymerase Chain Reaction) primers (10 mer), the T. matsutake FERM BP-7304 strain was compared with 44 kinds of known T. matsutake strains (for example, the IFO 6915 strain; Institute for Fermentation Osaka). The T. matsutake FERM BP-7304 strain exhibited a DNA pattern different from all of the other known T. matsutake strains (44 kinds).

Preferable embodiments of the cancer preventive agent and food of the present invention contain as an active ingredient: (i) fresh mushrooms of T. matsutake FERM BP-7304 strain (e.g., mycelia, broths, or fruit bodies of the strain) or a dried powder thereof; (ii) a hot water extract of the T. matsutake FERM BP-7304 strain (e.g., a hot water extract of mycelia, broths, or fruit bodies of the strain); or (iii) an alkaline solution extract of the T. matsutake FERM BP-7304 strain (e.g., an alkaline solution extract of mycelia, broths, or fruit bodies of the strain). However, the active ingredient is not limited to these embodiments.

For the present invention, the above embodiment (i) is preferable.

As mycelia of the T. matsutake FERM BP-7304 strain usable as the active ingredient of the cancer preventive agent and food of the present invention, mycelia may be used, for example, in a form obtained directly by removing a medium from a mixture of mycelia obtained by culturing (that is, cultured mycelia) and a medium with an appropriate removing means (e.g., filtration). Alternatively, dried mycelia, which are obtained by removing water from the mycelia after the removal of the medium with an appropriate removing means (e.g., lyophilization) may be used. Further, dried mycelia powders, which are obtained by grinding the above dried mycelia, may be used.

As broths of the T. matsutake FERM BP-7304 strain usable as the active ingredient of the cancer preventive agent and food of the present invention, a broth may be used, for example, in the form of a mixture of mycelia obtained by cultivation (that is, cultured mycelia) and a medium. Alternatively, a dried broth obtained by removing water from the above mixture with an appropriate removing means (e.g., lyophilization) may be used. Further, dried broth powders, which are obtained by grinding the above dried broth, may be used.

A method for the above-described cultivation is not particularly limited, and any of the ordinary methods for culturing T. matsutake fungi can be used. However, a method, for example, disclosed in JP Patent Application No. 2002-311840 is preferably employed, since the method enables mass production without the loss of the physiological activities of matsutake fungi. The method comprises: a step for obtaining matsutake fungi II by culturing or preserving the T. matsutake FERM BP-7304 strain (“matsutake fungi I”) in a solid or liquid medium; a step for obtaining matsutake fungi III by stationary liquid-cultivation of the matsutake fungi II; a step for obtaining matsutake fungi IV by shaking cultivation of the matsutake fungi III; a step for obtaining matsutake fungi V by stirring-culture of the matsutake fungi IV with the use of a small culture apparatus with a volume of less than 100 L without the aeration in a liquid medium; a step for obtaining matsutake fungi VI by deep stirring-culture of the matsutake fungi V with the use of a medium- or large-sized culture apparatus with a volume of 100 L or more; a step for obtaining matsutake fungi VII by deep stirring-culture of the matsutake VI with the use of a medium- or large-sized culture apparatus with a volume of 100 L or more; and a step for obtaining matsutake fungi VIII by deep stirring-culture of the matsutake fungi VII with the use of a medium- or large-sized culture apparatus with a volume of 100 L or more.

<Step for Obtaining matsutake Fungi II by Culturing or Preserving matsutake Fungi I>

A medium to be used herein is not particularly limited, as long as such medium is a common one containing a nutrient substrate for culturing matsutake fungi. Examples thereof include an Ohta medium (Ohta et al., “Trans. Mycol. Soc. Jpn.,” 31, 323-334, 1990), an MMN medium (Marx, D. H., “Phytopathology,” 59: 153-163, 1969), and a Hamada medium (Hamada, “Matsutake,” 97-100, 1964), but the usable medium is not limited to these examples.

Preferable examples of a solidifying agent for a solid medium include carrageenan, mannnan, pectin, agar, curdlan, starch, and alginic acid. Among these, agar is preferable.

Examples of usable nutrient substrate for a medium include a carbon source, a nitrogen source, and an inorganic element source.

Examples of the above carbon source include: starches, such as rice starch, wheat flour starch, potato starch, and sweet potato starch; polysaccharides, such as dextrin and amylopectin; oligosaccharides, such as maltose and sucrose; and monosaccharides, such as fructose and glucose. Examples thereof further include malt extracts. Depending on the growth speed of matsutake fungi, matsutake has a period in which monosaccharides such as glucose are preferably used and a period in which starches are preferably used. Therefore, a suitable carbon source is selected based on the period, and if necessary, these carbon sources may be used in combination.

Examples of the above nitrogen source include naturally occurring substances such as yeast extracts, dried yeast, corn steep liquor, soy flour, and soy peptone, ammonium nitrate, ammonium sulfate, and urea. These may be used either alone or in combination. In general, considering growth speed, naturally occurring substances, particularly yeast extracts, are preferable.

The inorganic element source is used to supply phosphoric acid and trace elements. Examples thereof include, in addition to phosphates, inorganic salts (e.g., sulfates, hydrochlorides, nitrates, and phosphates) of metal ions such as sodium, potassium, magnesium, calcium, zinc, manganese, copper, andiron. A required amount of the inorganic element is dissolved in a medium.

In addition, vitamins such as vitamin B₁ or amino acids may be added to the medium.

Further, in accordance with the properties of matsutake fungi to be used, plant extracts, organic acids, nucleic acid-related substances or the like may be added. Examples of the plant extracts include extracts of fruit crops, root crops, and leaf vegetables. Examples of the organic acids include citric acid, tartaric acid, malic acid, fumaric acid, and lactic acid. Examples of the nucleic acid-related substances include commercially available nucleic acids, nucleic acid extracts, yeast, and yeast extracts.

In preparing a solid medium, the amount of carbon source to be used is preferably 10 to 100 g/L, more preferably 10 to 50 g/L, and most preferably 20 to 30 g/L.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphate to be used is in phosphorus equivalent, preferably 0.001 to 0.05 mol/L, more preferably 0.005 to 0.03 mol/L, and most preferably 0.01 to 0.02 mol/L. In addition, other inorganic salts, vitamins, plant extracts, organic acids, nucleic acid-related substances, or the like may be optionally added in accordance with the properties of the matsutake fungi. Furthermore, the prepared nutrient substrate solution is adjusted so as to have a pH of preferably 4 to 7, more preferably 4.5 to 6.0, and most preferably 5.0 to 5.5.

<Stationary Liquid Cultivation>

Next, a method for producing matsutake fungi III by stationary cultivation of matsutake fungi II (matsutake fungi cultured or preserved in a solid or liquid medium) in a liquid medium will be described.

Usually, an Erlenmeyer flask with a volume of 100 mL to 2 L is used.

The stationary liquid cultivation starts by inoculating matsutake fungi II on the liquid medium.

The liquid medium is used, in which the ratio (“magnification at the time of inoculation”) of a mixture of the culture liquid containing the matsutake fungi II with a liquid medium to the culture liquid containing the matsutake fungi II is preferably 2:1 to 50:1, and more preferably 3:1 to 30:1.

The culture liquid containing the matsutake fungi II is inoculated on the liquid medium so that the ratio (“concentration of initial mycelia”) between the mass of dried mycelia of matsutake fungi II in the culture liquid containing the matsutake fungi II and the volume of the mixture of the culture liquid containing the matsutake fungi II with the liquid medium becomes preferably 0.05 to 3 g/L, and more preferably 0.1 to 2 g/L.

The temperature for the stationary liquid cultivation is preferably 15 to 30° C., and more preferably 20 to 25° C., and the cultivation period is preferably 30 to 400 days and more preferably 120 to 240 days. If the cultivation period is less than 30 days or more than 400 days, it is difficult to obtain matsutake fungi III having growth ability suitable for mass culture.

In terms of growth ability, the culturing is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The liquid medium to be used for the stationary liquid cultivation contains a nutrient substrate so that the medium has an osmotic pressure of preferably 0.01 to 0.8 MPa, more preferably 0.02 to 0.7 MPa, and most preferably 0.03 to 0.5 MPa.

As the nutrient source to be used for the stationary liquid cultivation, the same carbon source, nitrogen source, inorganic element source, vitamins such as vitamin B₁, amino acids, and the like can be used as those used for the solid medium for culturing matsutake fungi I.

The amount of carbon source to be used is preferably 10 to 100 g/L, more preferably 20 to 60 g/L, and most preferably 25 to 45 g/L. Generally, monosaccharides such as glucose are used.

The amount of nitrogen source to be used is in nitrogen equivalent, preferably 0.005 to 0.1 mol/L, more preferably 0.007 to 0.07 mol/L, and most preferably 0.01 to 0.05 mol/L.

When phosphates are used, the amount thereof to be used is in phosphorus equivalent, preferably 0.001 to 0.05 mol/L, more preferably 0.005 to 0.03 mol/L, and most preferably 0.01 to 0.02 mol/L.

In addition, other inorganic salts, vitamins, plant extracts, organic acids, nucleic acid-related substances, or the like may be properly added in accordance with the properties of matsutake fungi.

The prepared nutrient substrate solution has a pH of preferably 4 to 7, more preferably 4.5 to 6.5, and most preferably 5.0 to 6.0.

A part or the whole of the culture liquid containing matsutake fungi III by stationary liquid cultivation may be used again as an inoculation source for stationary liquid cultivation in the stationary liquid cultivation step in the same manner as the culture liquid (or culture product) containing matsutake fungi II.

<Shaking Cultivation>

Next, a method for producing matsutake fungi IV by shaking cultivation of matsutake fungi III will be described.

In general, an Erlenmeyer flask with a volume of 300 mL to 5 L is used.

The shaking cultivation starts by inoculating matsutake fungi III on a liquid medium.

The liquid medium is used, in which the ratio (“magnification at the time of inoculation”) of a mixture of the culture liquid containing the matsutake fungi III with a liquid medium to the culture liquid containing the matsutake fungi III is preferably 2:1 to 50:1, and more preferably 3:1 to 30:1.

Further, in order to secure enough amount of the culture liquid to meet the magnification at the time of inoculation, the stationary liquid culture may be produced using a plurality of culture apparatuses.

The culture liquid containing the matsutake fungi III is inoculated on the liquid medium so that the ratio (“concentration of initial mycelia”) between the mass of dried mycelia of matsutake fungi III in the culture liquid containing the inoculated matsutake fungi III and the volume of the mixture of the culture liquid containing the inoculated matsutake fungi III with the liquid medium becomes preferably 0.05 to 3 g/L, more preferably 0.1 to 2 g/L.

In the shaking cultivation, the temperature is preferably 15 to 30° C. and more preferably 20 to 25° C., and the culture period is preferably 7 to 50 days and more preferably 14 to 28 days.

As power required for the shaking culture, a power of 0.05 to 0.4 kW/m³ for shaking a unit volume of the culture liquid in the Erlenmeyer flask is generally used.

In terms of growth ability, the cultivation is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The liquid medium to be used for the shaking cultivation contains a nutrient substrate so that the medium has an osmotic pressure of preferably 0.01 to 0.8 MPa, more preferably 0.02 to 0.7 MPa, and most preferably 0.03 to 0.5 MPa.

As the nutrient source to be used for the shaking culture, the same carbon source, nitrogen source, inorganic element source, vitamins such as vitamin B₁, amino acids, and the like can be used as those used for the liquid medium for culturing matsutake fungi II.

The amount of carbon source to be used is preferably 10 to 100 g/L, more preferably 20 to 60 g/L, and most preferably 25 to 45 g/L. Generally, monosaccharides such as glucose are used.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphate salts to be used is in phosphorus equivalent, preferably 0.001 to 0.05 mol/L, more preferably 0.005 to 0.03 mol/L, and most preferably 0.01 to 0.02 mol/L.

In addition, other inorganic salts, vitamins, amino acids, plant extracts, organic acids, nucleic acid-related substances, or the like may be properly added in accordance with the properties of the matsutake fungi.

The prepared nutrient substrate solution has a pH of preferably 4 to 7, more preferably 4.5 to 6.5, and most preferably 5.0 to 6.0.

<Stirring Cultivation>

Next, a method for producing matsutake fungi V, matsutake fungi VI, matsutake fungi VII, and matsutake fungi VIII by stirring cultivation will be described.

The stirring cultivation starts by inoculating matsutake fungi (IV to VII) on a liquid medium.

The liquid medium to be used for the stirring cultivation is prepared in the following manner.

As a nutrient substrate, the same carbon source, nitrogen source, inorganic element source, vitamins such as vitamin B₁, and amino acids may be used as those used for the shaking cultivation.

The amount of carbon source to be used is preferably 10 to 100 g/L, more preferably 20 to 60 g/L, and most preferably 25 to 45 g/L. Starches are preferably used.

When monosaccharides such as glucose, which affects the osmotic pressure of the culture liquid to be stirred, are used in combination, the amount thereof to be used is preferably 0.1 to 60 g/L, more preferably 0.5 to 40 g/L, and most preferably 0.7 to 20 g/L.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphates to be used is in phosphorus equivalent, preferably 0.001 to 0.05 mol/L, more preferably 0.005 to 0.03 mol/L, and most preferably 0.01 to 0.02 mol/L.

Further, other inorganic salts, vitamins, amino acids, plant extracts, organic acids, nucleic acid-related substances, and the like may be properly added in accordance with the properties of matsutake fungi.

The pH of the prepared nutrient substrate solution is preferably 4 to 7, more preferably 4.5 to 6.5, and most preferably 5.0 to 6.0.

The liquid medium to be used for stirring cultivation contains a nutrient substrate so that it has an osmotic pressure of preferably 0.01 to 0.8 MPa, more preferably 0.02 to 0.7 MPa, and most preferably 0.03 to 0.5 MPa.

The temperature for the stirring cultivation is 15 to 30° C., preferably 20 to 25° C.

The liquid medium is used, in which the ratio (“magnification at the time of inoculation”) of a mixture of the culture liquid containing the matsutake fungi (IV to VII) with the liquid medium to the culture liquid containing the inoculated matsutake fungi (IV to VII) is preferably 2:1 to 50:1, more preferably 3:1 to 30:1, and most preferably 5:1 to 10:1.

The culture liquid containing the matsutake fungi (IV to VII) is inoculated on the liquid medium so that the volume ratio (“concentration of initial mycelia”) between the mass of dried mycelia of matsutake fungi (IV to VII) in the culture liquid containing inoculated matsutake fungi (IV to VII) and the mixture of the culture liquid containing the inoculated matsutake fungi (IV to VII) with the liquid medium becomes preferably 0.01 to 5 g/L, more preferably 0.05 to 3 g/L, and most preferably 0.1 to 2 g/L.

When matsutake fungi (V to VII) obtained by the stirring culture is used as mother fungi for stirring cultivation, the cultivation period is preferably 3 to 20 days, and particularly preferably 5 to 14 days.

After the cultivation period, the culture liquid contains matsutake fungi (V to VII), which have growth ability suitable for stirring cultivation, at amounts equivalent to dried mycelia content of preferably 0.5 to 10 g/L, more preferably 1 to 8 g/L, and most preferably 1 to 6 g/L.

In terms of growth ability, the culture is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The cultivation period for isolating matsutake mycelia from the matsutake fungi (V to VIII) obtained by the stirring cultivation is 5 to 30 days, more preferably 7 to 20 days, and most preferably 10 to 15 days.

During the above cultivation periods, the time when the assimilation speed of the carbon source decreases remarkably is considered to be the preferable time for terminating the cultivation. However, the time for terminating the cultivation can be properly determined in accordance with production patterns such as production cycle and production cost.

In terms of industrial production, the cultivation is preferably performed so that the dried mycelia content (unit: g/L) in the culture liquid after the stationary liquid cultivation becomes 35 to 100 times (in a ratio referred to as “mycelia increase ratio”) greater than the concentration of initial mycelia.

The culture liquid containing matsutake fungi IV produced by stirring cultivation may be used for a stirring cultivation step with the use of a culture apparatus such as a medium- or large-sized culture tank with a volume of 100 L or more.

The culture apparatus to be used for stirring cultivation is not particularly limited as long as the apparatus is capable of aeration-cultivation and maintaining sterility. As occasion demands, an apparatus that enables aeration or that can be installed with an aeration apparatus may be used. Therefore, an ordinary small-, medium-, and large-sized culture tank, or a jar fermentor, can be used.

In producing matsutake fungi V by culturing matsutake IV by the use of a jar fermentor or a small-sized culture tank with a volume of less than 100 L, the stirring cultivation is performed preferably without aeration in the liquid medium. The reason is that when the cultivation is performed with aeration in a jar fermentor or small-sized culture tank with a volume of less than 100 L, mycelia grow closely to each other to lose their growing points and their growing ability of mother fungi is damaged.

Further, when the cultivation with deep stirring is performed at industrial scale by the use of a culture apparatus such as a medium- or large-sized culture tank with a volume of 100 L or more, aeration is carried out when needed. In this case, the aeration volume is 0.05 to 1.0 vvm, and in particular preferably 0.2 to 0.5 vvm.

The stirring in the stirring cultivation is controlled by a stirring power required for a unit volume of the culture liquid at an early stage of the cultivation. Generally, by stirring within a power range of preferably 0.01 to 2 kW/m³ and more preferably 0.05 to 1 kW/m³, matsutake mycelia grow favorably. After the early stage, the fungi start to grow, thereby causing insufficient oxygen supply. Further, grown mycelia do not disperse adequately, and thus a larger strength of stirring is properly required. For the deep stirring, preferably, early stage cultivation is conducted with low aeration at low stirring speed and late stage cultivation is performed with high aeration at high stirring speed.

The separation and collection of matsutake mycelia obtained by the deep stirring cultivation may be carried out by conventional methods. Examples of these methods include filtration by a filter press or the like, and centrifugation.

The obtained mycelia are preferably washed well with, for example, distilled water, and then provided for the subsequent hot water extraction step. Further, in order to enhance the extraction efficiency, the mycelia are preferably processed into crushed materials or powders.

As the fruit bodies of the T. matsutake FERM BP-7304 strain usable as the active ingredient of the cancer preventive agent and food of the present invention, for example, fruit bodies as they are, or crushed fruit bodies, can be used. Alternatively, dried fruit bodies obtained by removing water therefrom with an appropriate removing means (e.g., lyophilization), may be used. Further, dried fruit body powders obtained by grinding the above dried fruit bodies may be used.

The hot water extract of the T. matsutake FERM BP-7304 strain usable as the active ingredient of the cancer preventive agent and food of the present invention can be prepared by, for example, extracting mycelia, (i.e., the cultured mycelia), broths, or fruit bodies obtained by culturing the T. matsutake FERM BP-7304 strain with hot water.

The temperature of hot water to be used for the hot water extraction is not particularly limited, as long as the component that is contained in the T. matsutake FERM BP-7304 strain and that exhibits cancer prevention activity is sufficiently extracted so as to result in the hot water extract. However, the temperature is preferably about 60 to 100° C., and more preferably about 80 to 98° C.

When mycelia or fruit bodies are used for the hot water extraction, it is preferable to process them into crushed materials or powders to enhance the extraction efficiency.

Further, it is preferable to carry out the hot water extraction step while stirring or shaking to improve the extraction efficiency. The period for extraction may be properly determined in accordance with, for example, the form of mycelia (e.g., a processed state when they are processed into a crushed or pulverized form), the temperature of the hot water, or treatment conditions with or without stirring or shaking. However, it is usually about 1 to 6 hours, and preferably about 2 to 3 hours.

The obtained hot water extract may be used as it is, namely, in a state containing insolubles, as the active ingredient of the cancer preventive agent of the present invention. Alternatively, it may be used as the active ingredient of the cancer preventive agent of the present invention after the insolubles and then low molecular weight fractions (preferably fractions containing substances with a molecular weight of 3500 or less) are removed from the extract.

The alkaline solution extract of the T. matsutake FERM BP-7304 strain usable as the active ingredient of the cancer preventive agent and food of the present invention may be prepared by, for example, a method similar to the above-mentioned method for preparing the hot water extract of T. matsutake FERM BP-7304 strain, except that an alkaline solution is used instead of hot water.

An alkaline solution to be used for the alkaline solution extraction is not particularly limited, but, for example, hydroxides of alkaline metals (sodium, potassium, etc.), and in particular an aqueous solution of sodium hydroxide, may be used. The alkaline solution preferably has a pH of 8 to 13, and more preferably 9 to 12. The alkaline solution extraction is conducted preferably at a temperature of about 0 to 30° C., more preferably about 0 to 25° C. A period for extraction may be properly determined in accordance with, for example, the state of the mycelia residue (e.g., a processed state when the mycelia are processed into a crushed or pulverized form), the pH value or the temperature of the alkaline solution, or treatment conditions with or without stirring or shaking, but it is usually about 30 minutes to 5 hours, and preferably about 1 to 3 hours. The obtained alkaline solution extract may be directly used, or, if desired, subjected to neutralization treatment, and then used for the cancer preventive agent and food of the present invention.

The cancer preventive agent and food of the present invention can be administered to animals or humans, having as the active ingredient T. matsutake, in particular the T. matsutake FERM BP-7304 strain, or an extract thereof, either alone or, if desired, in combination with a pharmaceutically acceptable carrier.

In the present invention, the expression “cancer prevention” means preventing the occurrence of cancer in animals or humans, and further, when so-called precancerous symptoms are observed, the expression means effects of delaying or inhibiting malignant transformation of precancerous states. Therefore, the administration or intake timing of the cancer preventive agent and food of the present invention is not particularly limited, but it is preferable to routinely conduct continuous administration or intake thereof.

The cancer prevention effects of the present invention are not dependent on the type of cancer, and therefore the effects of the present invention can be obtained regarding various cancers such as cancers of the colon, lung, breast, prostate, esophagus, stomach, and liver. In particular, the present invention is excellent in preventing the occurrence of colon cancer.

The formulation for administration and intake of the cancer preventive agent and food of the present invention is not particularly limited to, but may be, for example, oral medicines such as powders, fine particles, granules, tablets, capsules, suspensions, emulsions, syrups, extracts or pills, or parenteral medicines such as injections, liquids for external use, ointments, suppositories, creams for topical application, or eye lotions.

The oral medicines may be prepared by conventional methods using, for example, fillers, binders, disintegrating agents, surfactants, lubricants, flowability-enhancers, diluting agents, preservatives, coloring agents, perfumes, tasting agents, stabilizers, humectants, antiseptics, and antioxidants. Examples of the aforementioned include gelatin, sodium alginate, starch, corn starch, saccharose, lactose, glucose, mannitol, carboxylmethylcellulose, dextrin, polyvinyl pyrrolidone, crystalline cellulose, soybean lecithin, sucrose, fatty acid esters, talc, magnesium stearate, polyethylene glycol, magnesium silicate, silicic anhydride, and synthetic aluminum silicate.

The parenteral administration may take the form of, for example, an injection such as a subcutaneous or intravenous injection, or rectal administration. Among the parenteral formulations, an injection is preferably used.

In preparing injections, for example, water-soluble solvents, such as physiological saline or Ringer's solution, water-insoluble solvents, such as plant oil or fatty acid esters, isotonizing agents such as glucose or sodium chloride, solubilizing agents, stabilizing agents, antiseptics, suspending agents, or emulsifying agents may be optionally used, in addition to the active ingredient.

The cancer preventive agent and food of the present invention may be administered in the form of a sustained release preparation using sustained release polymers. For example, the cancer preventive agent and food of the present invention may be incorporated in a pellet made of ethylenevinyl acetate polymers, and the pellet may be surgically implanted in a tissue to be treated or which is to be protected from cancer.

The cancer preventive agent and food of the present invention contain as the active ingredient T. matsutake FERM BP-7304 strain or extracts thereof, or the like in amounts of 0.01 to 99% by mass, and preferably 0.1 to 90% by mass. However, amounts are by no means limited to the aforementioned.

A dose for administration or intake of the cancer preventive agent and food of the present invention may be properly determined depending on the kind of disease, the age, sex, body weight, symptoms of a patient, method of administration or intake. The cancer preventive agent and food of the present invention may be orally or parenterally administered or taken.

The form of administration or intake is not limited to a medicament, but various forms are available, such as eatable or drinkable products such as health-promoting foods (specified health foods and nutritional-functional foods), as so-called health foods (both including drinkable products), or as feeds. Further, the cancer preventive agent and food of the present invention may be administered in the form of an agent that is temporarily kept in the mouth, but then spat out without the retention of most components, for example, a dentifrice, a mouthwash agent, a chewing gum, or a collutorium, or in the form of an inhalant drawn in through the nose. For example, the active ingredient such as T. matsutake FERM BP-7304 strain or extracts thereof may be added to a desired food (including a drink), a feed, a dentifrice, a mouthwash agent, a chewing gum, a collutorium, or the like as an additive (such as a food additive).

In the above description, the term “specified health food” means a food, for which it is permitted to indicate health functions possessed by that food (permission by Ministry of Health, Labor, and Welfare is required for each food). The term “nutritional-functional food” means a food, for which it is allowed to explicitly state the functions of nutritional components (the standard prescribed by Ministry of Health, Labor, and Welfare should be satisfied). The term “health food” widely means foods in general other than the above-mentioned health-promoting foods, and health food includes health supplements.

### EXAMPLES

The present invention will be described in detail by referring to the following Examples, but the technical scope of the present invention is not limited by these Examples.

### Example 1

[Preparation of Dry Powder (Hereinafter also Referred to as “CM6271”) of Mycelia of the T. matsutake FERM BP-7304 Strain]

Mycelia of the T. matsutake FERM BP-7304 strain were inoculated into a 7-ton culture tank containing 3.5 tons of sterilized medium (3% glucose, 0.3% yeast extract, pH 6.0), and cultured while being stirred at 25° C. for 4 weeks. The obtained culture product was filtrated with filter cloth, and after mycelia were separated they were washed thoroughly with distilled water.

A portion (ca. 1 kg) of the obtained mycelia was frozen at −60° C., and then lyophilized using a lyophilizer (MINIFAST MOD. DO. 5; Edwards), so that 110 g of dried mycelia was obtained.

The obtained mycelia were ground using a homoblender (Wonder Blender), and thereby 100 g of dried powder (CM6271) was obtained.

### Example 2

[Inhibition of Rat Colon Precancerous Changes Induced by Azoxymethane (AOM)]

(i) Test Animal

6-week old F344 male rats were purchased from Japan SLC, Inc. The rats were accommodated in polycarbonate cages in a safe and clean rack in an infection experiment animal room, and bred at temperatures of 23±2° C. and humidity of 55±10% under an environment with luminary air flow and with a photopeirod of from 8:00 to 20:00 with free provision of feed CE-2 (Oriental Yeast Co., Ltd.) and sterilized tap water. These rats were quarantined and inspected, and thereafter pre-bred for 1 week (to result in 7-week old rats). To keep a constant breeding environment, only keepers and experimenters were permitted to enter the animal room.

(ii) Reagent

Azoxymethane (AOM), which is known as a carcinogen, was used. Methylene blue was used as a color reagent. Both were purchased from Sigma Chem. Co. (U.S.).

(iii) Preparation of CM6271-Added Feed

To powder feed CE-2, CM6271 (dried powder of mycelia of the T. matsutake FERM BP-7304 strain) obtained in Example 1 was added at ratios of 0.125%, 0.5%, or 2.0%, and the feed to which CM6271 had been added was prepared before use.

(iv) Experiment Group Constitution and Treatment

7-week old F344 male rats were divided at random into the following 4 groups (n=10).

(1) Control group to which ordinary feed (only CE-2 powder) was provided

(2) 0.125% CM6271 added-feed intake group

(3) 0.5% CM6271 added-feed intake group

(4) 2.0% CM6271 added-feed intake group

One week after the initiation of the experiment, 15 mg/kg of AOM was subcutaneously injected once a week, a total of 3 times. During the experiment period, the weight and the amount of feed intake were measured every week.

(v) Measurement of Precancerous Changes

7 weeks after the initiation of the AOM treatment, the rats were sacrificed and their colons were removed. After these colons were thoroughly washed with physiological saline, they were incised from the anus to the cecum using dissection scissors. They were fixed on a rubber plate in such manner that the mucosa thereof was turned upward, and then soaked in 10% formalin-PBS solution for 24 hours. Next, the resultant colon was placed in flowing water for 30 minutes or more to remove formalin, and thereafter soaked in 0.2% methylene blue solution for 10 minutes for staining. After the staining, attached pigments were removed in flowing water and then the numbers of the aberrant crypt foci (ACF) and aberrant crypts (AC) were measured using a stereoscopic microscope. The identification and count of ACF was carried out in accordance with the Bird method (Bird RP: Observation and quantification of aberrant crypt foci in the murine colon treated with a colon carcinogen: preliminary findings, “Cancer Letters,” 37: 147-151, 1987)

(vi) Serologic Test and Organ Weight Measurement

After the experiment, blood was collected and a blood test was conducted, and the weight of the liver was also measured.

(vii) Statistical Analysis

A significant difference test was conducted by Student t-test, and “p<0.05” was determined to be significant.

(viii) Results and Considerations

(a) Results of Changes in Body Weight and Feed Intake Amount

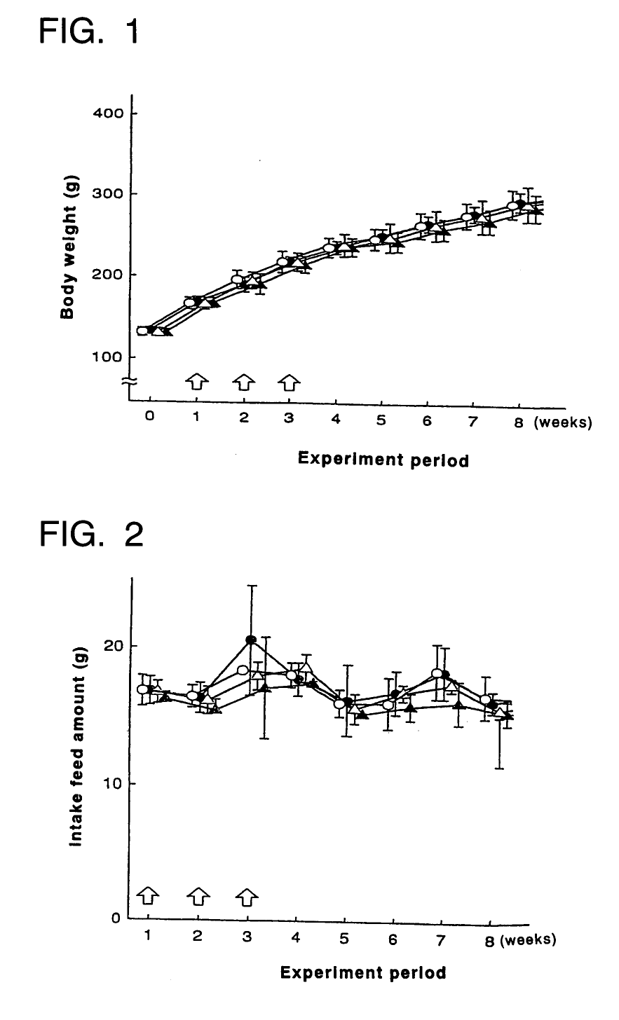

FIGS. 1 and 2 show chronological changes of body weights and feed intake amounts, respectively. In FIGS. 1 and 2, white circles, black circles, white triangles, and black triangles represent, respectively, the control group, the 0.125% CM6271-added feed group, the 0.5% CM6271-added feed group, and the 2.05% CM6271-added feed group. In addition, in FIGS. 1 and 2, white up-arrows (first, second, and third weeks after the initiation of experiment) indicate the timing when AOM was injected.

As is clear from FIG. 1, 4 groups steadily increased their body weights during the 8-week experiment period and almost no influence of CM6271 was observed. Further, as is clear from FIG. 2, almost no influence of the addition of CM6271 was observed on the amounts of feed intake.

(b) Inhibition of Precancerous Changes Due to Intake of CM6271-Added Feed

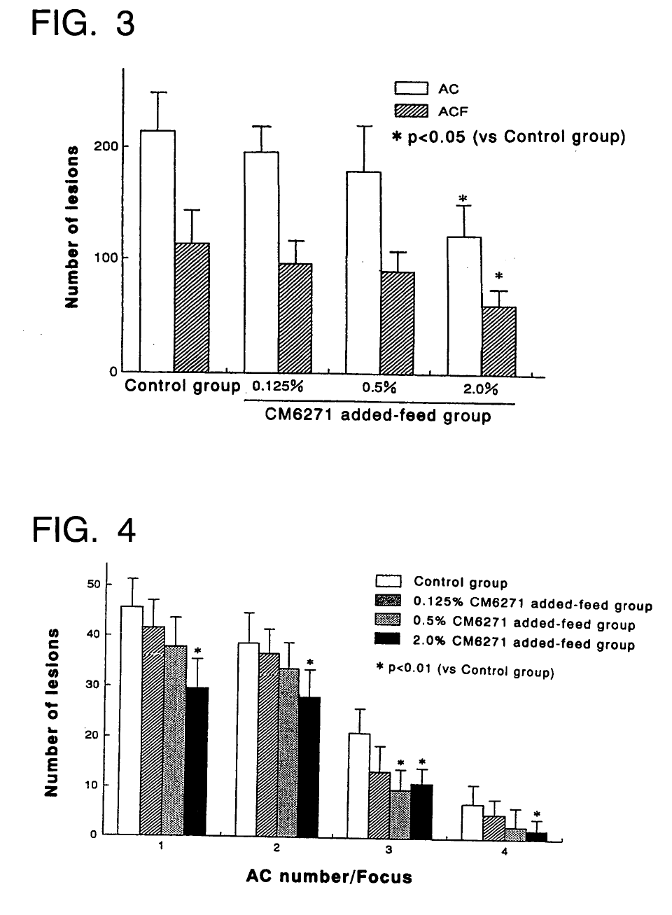

FIG. 3 is a graph showing precancerous changes due to intake of CM6271-added feed.

<ACF Number>

As shown in FIG. 3, the control group had an ACF number of 115±28. In contrast, the 0.125% CM6271-added feed group, the 0.5% CM6271-added feed group, and the 2.0% CM6271-added feed group had ACF numbers of 99±17, 89±18, and 58±11, respectively. CM6271 dose-dependently inhibited the ACF number. Further, significant differences between the control group and the 2.0% CM6271-added group were found.

<AC Number>

Furthermore, the control group had an AC number of 219±32. In contrast, the 0.125% CM6271-added feed group, the 0.5% CM6271-added feed group, and the 2.0% CM6271-added feed group had AC numbers of 196±23, 178±38, and 128±29, respectively. CM6271 dose-dependently inhibited the AC number. Further, significant differences between the control group and the 2.0% CM6271-added group were found.

(c) Influence of the CM6271-Added Feed Intake on AC Number per Focus

Next, the influence of CM6271 administration on AC number per focus was examined. The results are shown in FIG. 4. As shown in the figure, the 2.0% CM6271-added feed group exhibited remarkable inhibition effects on lesions with an AC number of 1 to 2 in comparison with the control group. Thus, it is considered that CM6271 acts at a relatively early stage of carcinogenesis.

(d) Organ Weight at Autopsy and Hematological Findings

Table 1 shows liver weights at autopsy and ratios between liver weights and body weights. All 4 groups were at almost the same level in terms of the liver weight and the ratio between liver weight and body weight, and almost no influence of CM6271 was observed.

Further, as shown in Table 2, almost no influence of CM6271 was observed in terms of hematological findings.

TABLE 1

| | | | Liver weight/ |
|---|---|---|---|
| Group (n = 10) | Body weight | Liver weight | Body weight |
| Control group | 326 ± 20 g | 11.4 ± 1.2 g | 3.48 ± 0.29 |
| 0.125% CM6271-added | 331 ± 12 g | 11.9 ± 0.7 g | 3.59 ± 0.23 |
| feed group | | | |
| 0.5% CM6271-added | 332 ± 26 g | 11.7 ± 0.7 g | 3.50 ± 0.40 |
| feed group | | | |
| 2.0% CM6271-added | 320 ± 17 g | 11.6 ± 1.5 g | 3.61 ± 0.41 |
| feed group | | | |

TABLE 2

| | White | Red | | | | | | |
|---|---|---|---|---|---|---|---|---|
| | blood | blood | | | | | | |
| Group (n = 10) | cell | cell | HCB | HCT | MCV | MCH | MCHC | PLT |
| Control group | 110 ± 6 | 1087 ± 37 | 16.6 ± 0.5 | 52.6 ± 1.7 | 48.4 ± 1.1 | 15.3 ± 0.8 | 31.6 ± 1.3 | 87 ± 14 |
| 0.125% | 125 ± 9 | 1114 ± 60 | 17.3 ± 0.5 | 53.2 ± 2.6 | 47.6 ± 0.9 | 15.5 ± 0.5 | 32.5 ± 1.0 | 88 ± 9 |
| CM6271-added | | | | | | | | |
| feed group | | | | | | | | |
| 0.5% | 111 ± 13 | 1128 ± 41 | 17.3 ± 1.9 | 53.5 ± 0.6 | 47.4 ± 0.5 | 15.4 ± 0.8 | 32.4 ± 1.7 | 79 ± 3 |
| CM6271-added | | | | | | | | |
| feed group | | | | | | | | |
| 2.0% | 125 ± 14 | 1106 ± 35 | 17.1 ± 0.7 | 52.2 ± 1.9 | 47.0 ± 0.0 | 15.4 ± 0.7 | 32.7 ± 1.5 | 87 ± 5 |
| CM6271-added | | | | | | | | |
| feed group | | | | | | | | |

In Table 2, units for individual columns are:
White blood cell: ×10²/mm³,
Red blood cell: ×10⁴/mm³,
HHCB: g/dl,
HCT: %,
MCV: μ³,
MCH: pg,
MCHC: %, and
PLT: ×10⁴/mm³.

The above results have suggested that the Tricholoma matsutake FERM BP-7304 strain has inhibition effects on AOM-induced colon precancerous changes.

It has been reported that krestin (PSK), Polyprus frondosus, and the like among mushrooms have cancer prevention activities, and it is considered that immunoregulation or antioxidant action is complicatedly involved in the obtainment of the effects. As an inhibition mechanism of AOM-induced colon precancerous changes according to the present research, it is considered that the Tricholoma matsutake FERM BP-7304 strain may affect AOM metabolism in the colon and inhibit the production of carcinogenic metabolites.

As described above in detail, the cancer preventive agent and food of the present invention can prevent the occurrence of cancer; specifically, cancer that is thought to be induced by azoxymethane. In particular, it can prevent the occurrence of colon cancer.

What is claimed is:

1. A cancer preventive agent containing Tricholoma matsutake or an extract thereof.

2. The cancer preventive agent according to claim 1, wherein Tricholoma matsutake is a mycelium, a broth, or a fruit body (including a spore).

3. The cancer preventive agent according to claim 1, wherein Tricholoma matsutake is the FERM BP-7304 strain.

4. The cancer preventive agent according to claim 1, wherein Tricholoma matsutake is dried powder of the mycelium of the FERM BP-7304 strain.

5. The cancer preventive agent according to claim 1, wherein the extract of Tricholoma matsutake is a hot water extract or an alkaline solution extract of the mycelium of the FERM BP-7304 strain.

6. The cancer preventive agent according to claim 1, wherein the agent is a colon cancer preventive agent.

7. A method of preventing a cancer which comprises administrating to a human or an animal in an effective amount of the cancer preventive agent of any one of claims 1-6.

8. A cancer preventive food containing Tricholoma matsutake or an extract thereof.

9. The food according to claim 8, wherein Tricholoma matsutake is a mycelium, a broth, or a fruit body (including a spore).

10. The food according to claim 8, wherein Tricholoma matsutake is the FERM BP-7304 strain.

11. The food according to claim 8, wherein Tricholoma matsutake is dried powder of the mycelium of the FERM BP-7304 strain.

12. The food according to claim 8, wherein the extract of Tricholoma matsutake is a hot water extract or an alkaline solution extract of the mycelium of the FERM BP-7304 strain.

13. The food according to claim 8, wherein the food is a colon cancer preventive agent.

14. A method of preventing a cancer which comprises the intake of by a human or an animal in an effective amount of the cancer preventive food of any one of claims 8-13.
